(19) United States
(12) Patent Application Publication
(10) Pub. No.: US 2021/0298731 A1
(43) Pub. Date: Sep. 30, 2021

(54) TAPERED ELONGATE SHAFT FOR MEDICAL DEVICE DELIVERY SYSTEMS

(71) Applicant: Covidien LP, Mansfield, MA (US)

(72) Inventors: Syamala Rani Pulugurtha, Irvine, CA (US); Ujwal Jalgaonkar, Irvine, CA (US); Edwin Bon, Lake Elsinore, CA (US); Komonn Lim, Lake Forest, CA (US); Maria Sanson, Mission Viejo, CA (US); Anthony Huynh, Wildomar, CA (US)

(21) Appl. No.: 17/249,249

(22) Filed: Feb. 24, 2021

Related U.S. Application Data
(60) Provisional application No. 63/002,176, filed on Mar. 30, 2020.

(51) Int. Cl. A61B 17/00; A61B 17/34

(52) U.S. Cl. CPC A61B 17/00234; A61B 2017/1205; A61B 17/3468

(57) ABSTRACT

An elongate shaft and associated systems and methods are disclosed herein. In some embodiments, the elongate shaft comprises a lumen extending along a length of the elongate shaft, and a structure radially peripheral to the lumen. The structure can be tapered in a distal direction such that an outermost cross-sectional dimension at a proximal portion of the structure is greater than an outermost cross-sectional dimension at a distal portion of the structure. An innermost cross-sectional dimension of the structure can be generally constant along all or a portion of the length of the elongate shaft. In some embodiments, the structure can comprise a plurality of layers or regions.

### CROSS-REFERENCE TO RELATED APPLICATION(S)

The present application claims the benefit of priority to U.S. Provisional Patent Application No. 63/002,176, filed Mar. 30, 2020, which is incorporated by reference herein in its entirety.

### TECHNICAL FIELD

The present technology relates to systems, devices, and methods for delivering medical devices to a treatment site.

### BACKGROUND

A medical catheter defining at least one lumen has been proposed for use with various medical procedures. For example, in some cases, a medical catheter may be used to access and treat defects in blood vessels, such as, but not limited to, lesions or occlusions in blood vessels.

### SUMMARY

The subject technology is illustrated, for example, according to various aspects described below, including with reference to FIGS. 1A-4. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., Clause 1, Clause 37, Clause 42, etc. The other clauses can be presented in a similar manner.

Clause 1. An elongate shaft for delivering a medical device, comprising:
a lumen extending along a length of the elongate shaft; and
a structure surrounding the lumen along the length of the elongate shaft, wherein an outermost surface of the structure is tapered in a distal direction such that an outermost cross-sectional dimension at a proximal portion of the structure is greater than an outermost cross-sectional dimension at a distal portion of the structure, and wherein the structure comprises an innermost cross-sectional dimension extending substantially along the length of the elongate shaft.
Clause 2. The elongate shaft of any one of the Clauses herein, wherein the structure comprises:
a first innermost region proximate the lumen; and
a second outermost region radially outward of the first region, the second region comprising the outermost surface of the structure.
Clause 3. The elongate shaft of any one of the Clauses herein, wherein the structure comprises:
a first region proximate the lumen;
a second region radially outward of the first region; and
a third region radially between the first and second regions.

Clause 4. The elongate shaft of any one of the Clauses herein, wherein the structure comprises:

a first region proximate the lumen;
a second region radially outward of the first region;
a third region radially between the first and second regions; and
a fourth region radially between the second and third regions.
Clause 5. The elongate shaft of any one of the Clauses herein, wherein the structure comprises:
a first region proximate the lumen;
a second region radially outward of the first region;
a third region radially between the first and second regions;
a fourth region radially between the second and third regions; and
a fifth region radially between the third and fourth regions.
Clause 6. The elongate shaft of any one of the Clauses herein, wherein the first region comprises at least one of a lubricious material, an elastomer, a polyolefin elastomer, a low durometer polyolefin elastomer, a synthetic fluoropolymer, polytetrafluoroethylene (PTFE), a migratory slip agent, and/or a permanent slip agent.
Clause 7. The elongate shaft of any one of the Clauses herein, wherein the second region comprises at least one of polymer tubing and/or braided tubing.
Clause 8. The elongate shaft of any one of the Clauses herein, wherein the second region comprises a stiffness that varies along at least a portion of the length of the elongate shaft.
Clause 9. The elongate shaft of any one of the Clauses herein, wherein the third region comprises a coiled or helical structure.
Clause 10. The elongate shaft of any one of the Clauses herein, wherein the third region comprises a coiled structure having a coil pitch that varies along the length of the elongate shaft.
Clause 11. The elongate shaft of any one of the Clauses herein, wherein the third region comprises a coiled structure having a coil pitch that decreases distally along the length of the elongate shaft.
Clause 12. The elongate shaft of any one of the Clauses herein, wherein the third region comprises Nitinol, titanium, stainless steel, cobalt, chromium, fiber, drawn filled tubing, and/or alloys thereof.
Clause 13. The elongate shaft of any one of the Clauses herein, wherein the fourth region comprises Nitinol, titanium, stainless steel, cobalt, chromium, fiber, drawn filled tubing, and/or alloys thereof.
Clause 14. The elongate shaft of any one of the Clauses herein, wherein the fifth region comprises a multi-layered polymer including a first innermost layer having a first stiffness, and a second outermost layer peripheral to the first layer and having a second stiffness, the second stiffness being stiffer than the first stiffness.
Clause 15. The elongate shaft of any one of the Clauses herein, wherein the multi-layered polymer further comprises a third layer radially between the first and second layers of the multi-layered polymer, the third layer having a third stiffness stiffer than the first stiffness and less stiff than the second stiffness.
Clause 16. The elongate shaft of any one of the Clauses herein, wherein each of the first region, second region, third region, fourth region and fifth region comprises at least one of a lubricious material, elastomer, synthetic fluoropolymer, polymeric tubing, braided tubing or fiber.
Clause 17. The elongate shaft of any one of the Clauses herein, wherein the first region comprises a slip agent and a polymer with low lubricity.
Clause 18. The elongate shaft of any one of the Clauses herein, wherein the first region, second region, third region, fourth region and fifth region comprise a first layer, a second layer, a third layer, a fourth layer, and a fifth layer, respectively.
Clause 19. The elongate shaft of any one of the Clauses herein, wherein an innermost cross-sectional dimension of the elongate shaft is substantially constant along a distal portion of the elongate shaft.
Clause 20. The elongate shaft of any one of the Clauses herein, wherein the innermost cross-sectional dimension of the elongate shaft is substantially constant along the entire length of elongate shaft.
Clause 21. The elongate shaft of any one of the Clauses herein, wherein the elongate shaft includes a longitudinal axis, wherein the structure includes a first portion extending along the longitudinal axis, a second portion extending along the longitudinal axis and distal to the first portion, and a third portion extending along the longitudinal axis and distal to the second portion, and wherein an outermost cross-sectional dimension of the third portion is less than an outer cross-sectional dimension of the first portion.
Clause 22. The elongate shaft of any one of the Clauses herein, wherein the second portion is tapered.
Clause 23. The elongate shaft of any one of the Clauses herein, wherein the outermost cross-sectional dimension of the second portion tapers in a distal direction.
Clause 24. The elongate shaft of any one of the Clauses herein, wherein the outermost cross-sectional dimensions of the first and third regions are substantially constant along the longitudinal axis.
Clause 25. The elongate shaft of any one of the Clauses herein, wherein the innermost cross-sectional dimension is at least about 0.017″, 0.021″, 0.027″, or 0.033″.
Clause 26. The elongate shaft of any one of the Clauses herein, wherein the innermost cross-sectional dimension is no more than about 0.017″, 0.021″, 0.027″, or 0.033″.
Clause 27. The elongate shaft of any one of the Clauses herein, wherein the innermost cross-sectional dimension is between about 0.017″ and 0.033″.
Clause 28. The elongate shaft of any one of the Clauses herein, wherein the outermost cross-sectional dimension at the proximal portion of the structure is at least about 1.6 French (Fr), 1.8 Fr, 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr.
Clause 29. The elongate shaft of any one of the Clauses herein, wherein the outermost cross-sectional dimension at the proximal portion of the structure is no more than about 1.6 French (Fr), 1.8 Fr, 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr.
Clause 30. The elongate shaft of any one of the Clauses herein, wherein the outermost cross-sectional dimension at the distal portion is at least about 1.8 French (Fr), 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr.
Clause 31. The elongate shaft of any one of the Clauses herein, wherein the outermost cross-sectional dimension at the distal portion is no more than about 1.8 French (Fr), 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr.
Clause 32. The elongate shaft of any one of the Clauses herein, wherein the outermost cross-sectional dimension at the distal portion is at least about 0.027″.
Clause 33. The elongate shaft of any one of the Clauses herein, wherein the outermost cross-sectional dimension at the proximal portion is at least about 0.039″.
Clause 34. The elongate shaft of any one of the Clauses herein, wherein the structure defines the lumen.
Clause 35. The elongate shaft of any one of the Clauses herein, wherein the elongate shaft comprises only the lumen and the structure.
Clause 36. The elongate shaft of any one of the Clauses herein, wherein the elongate shaft is a microcatheter.
Clause 37. A medical device delivery system, comprising:
an elongate shaft sized for insertion into a corporeal lumen, the elongate shaft comprising the elongate shaft of any one of the Clauses herein.
Clause 38. The medical device delivery system of any one of the Clauses herein, further comprising an implant or stent coupled to the elongate shaft, the elongate shaft being configured to advance the implant or stent through neurovasculature.
Clause 39. The medical device delivery system of any one of the preceding Clauses, wherein the elongate shaft is a second elongate shaft, the medical device delivery system further comprising a first elongate shaft including a lumen configured to receive the second elongate shaft.
Clause 40. The medical device delivery system of any one of the preceding Clauses, wherein the first elongate shaft is an aspiration catheter.
Clause 41. The medical device delivery system of any one of the preceding Clauses, wherein the lumen of the first elongate shaft has an innermost cross-sectional dimension of at least 0.050″ or 0.060″.
Clause 42. A method of manufacturing a medical delivery device, the method comprising:
providing an elongate shaft comprising a lumen extending along a length of the elongate shaft, and a structure surrounding the lumen along the length of the elongate shaft, the structure comprising an innermost cross-sectional dimension extending substantially along the length of the elongate shaft; and
altering portions of the elongate shaft such that an outermost cross-sectional dimension the structure is tapered in a distal direction.
Clause 43. The method of any one of the Clauses herein, wherein the elongate shaft is the elongate shaft of any one of the Clauses herein.
Clause 44. The method of any one of the Clauses herein, wherein altering comprises removing portions of the elongate shaft via chemically etching such that an outermost cross-sectional dimension at a proximal portion of the structure is greater than an outermost cross-sectional dimension at a distal portion of the structure.
Clause 45. The method of any one of the Clauses herein, wherein altering comprises removing portions of the elongate shaft via laser ablating such that an outermost cross-sectional dimension at a proximal portion of the structure is greater than an outermost cross-sectional dimension at a distal portion of the structure.
Clause 46. The method of any one of the Clauses herein, wherein altering comprises removing portions of the elongate shaft via grinding such that an outermost cross-sectional dimension at a proximal portion of the structure is greater than an outermost cross-sectional dimension at a distal portion of the structure.
Clause 47. The method of any one of the Clauses herein, wherein altering comprises removing portions of the elongate shaft via microblasting such that an outermost cross-sectional dimension at a proximal portion of the structure is greater than an outermost cross-sectional dimension at a distal portion of the structure.
Clause 48. The method of any one of the Clauses herein, wherein altering comprises joining two or more materials together such that an outermost cross-sectional dimension at a proximal portion of the structure is greater than an outermost cross-sectional dimension at a distal portion of the structure.
Clause 49. The method of any one of the Clauses herein, wherein joining two or more materials comprises laser welding, soldering, resistance welding, or thermal gluing the two or more materials.
Clause 50. A method of operating a medical device delivery system, the method comprising:
inserting the elongate shaft of any one of the Clauses herein into a lumen of a catheter; and
advancing the catheter through a corporeal lumen of a patient.
Clause 51. The method of any one of the Clauses herein, further comprising advancing the elongate shaft through the catheter such that a distal end portion of the elongate shaft is distal to a distal end of the catheter, and wherein an outermost cross-sectional dimension of the distal end portion of the elongate shaft is tapered relative to another portion of elongate shaft positioned within the catheter.
Clause 52. An elongate shaft for delivering a medical device, comprising:
a lumen extending along a length of the elongate shaft; and
a structure surrounding the lumen along the length of the elongate shaft, the structure comprising a first portion extending longitudinally from a proximal portion of the structure, a second portion extending longitudinally from the first portion, and a third portion extending longitudinally from the second portion to a distal portion of the structure, wherein an outermost surface of the structure is tapered in a distal direction such that an outermost cross-sectional dimension at the proximal portion of the structure is greater than an outermost cross-sectional dimension at the distal portion of the structure, and wherein the outermost cross-sectional dimension at the distal portion of the structure is the minimum outermost cross-sectional dimension of the structure.
Clause 53. The elongate shaft of any one of the Clauses herein, wherein the structure comprises a first innermost region proximate the lumen; and a second outermost region radially outward of the first region, the second region comprising the outermost surface of the structure, and the first and second regions extending longitudinally along the length of the elongate shaft.
Clause 54. The elongate shaft of any one of the Clauses herein, wherein the structure comprises:
a first region proximate the lumen and extending longitudinally along the length of the elongate shaft;
a second region radially outward of the first region and extending longitudinally along the length of the elongate shaft; and
a third region radially between the first and second regions and extending longitudinally along the length of the elongate shaft.
Clause 55. The elongate shaft of any one of the Clauses herein, wherein the first region, second region, and third region each comprise a layer, and wherein each of the first region, second region, and third region have a tapered portion.
Clause 56. The elongate shaft of any one of the Clauses herein, wherein the first region comprises a lubricious material, the second region comprises polymeric or braided tubing, and the third region comprises a coiled structure.
Clause 57. The elongate shaft of any one of the Clauses herein, wherein the third region comprises Nitinol, titanium, stainless steel, cobalt, chromium, fiber, or an alloy thereof.
Clause 58. The elongate shaft of any one of the Clauses herein, wherein the structure comprises:
a first region proximate the lumen;
a second region radially outward of the first region;
a third region radially between the first and second regions; and
a fourth region radially between the second and third regions.
Clause 59. The elongate shaft of any one of the Clauses herein, wherein the fourth region comprises a stiffness that varies in a distal direction.
Clause 60. The elongate shaft of any one of the Clauses herein, wherein the fourth region comprises a plurality of layers, wherein each of the layers has a stiffness different than that of the other layers.
Clause 61. The elongate shaft of any one of the Clauses herein, wherein the fourth region comprises a first layer having a first stiffness, a second layer radially peripheral to the first layer and having a second stiffness, and a third layer radially peripheral to the second layer and having a third stiffness, the second stiffness being stiffer than the first stiffness and the third stiffness being stiffer than the second stiffness.
Clause 62. The elongate shaft of any one of the Clauses herein, wherein the structure comprises:
a first region proximate the lumen;
a second region radially outward of the first region;
a third region radially between the first and second regions;
a fourth region radially between the second and third regions; and
a fifth region radially between the second and fourth regions,
wherein each of the first, second, third, fourth, and fifth regions extends along at least a majority of the length of the elongate shaft.
Clause 63. The elongate shaft of any one of the Clauses herein, wherein the distal portion is the distal terminus of the elongate shaft.
Clause 64. The elongate shaft of any one of the Clauses herein, wherein the second portion is tapered and the first and third portions are not tapered.
Clause 65. The elongate shaft of any one of the Clauses herein, wherein (i) the second portion is tapered, (ii) the first and third portions are not tapered, and (iii) the second portion is spaced apart from a distal terminus of the elongate shaft by no more than about 20 centimeters.
Clause 66. The elongate shaft of any one of the Clauses herein, wherein the lumen includes a substantially constant cross-sectional dimension along the length of the elongate shaft.
Clause 67. An elongate shaft for delivering a medical device, comprising:
a lumen extending along a length of the elongate shaft and having a substantially constant cross-sectional dimension along the length; and
a structure surrounding the lumen along the length of the elongate shaft, the structure comprising a first region extending longitudinally along the length of the elongated shaft, a second region radially outward of the first region, and a third region radially outward of the second region,
wherein an outermost surface of the structure is tapered in a distal direction such that an outermost cross-sectional dimension at a proximal portion of the structure is greater than an outermost cross-sectional dimension at a distal portion of the structure, and
wherein the outermost cross-sectional dimension at the distal portion of the structure is the minimum outermost cross-sectional dimension of the structure.
Clause 68. The elongate shaft of any one of the Clauses herein, wherein the third region comprises a stiffness that varies in a distal direction.
Clause 69. The elongate shaft of any one of the Clauses herein, wherein the third region comprises a plurality of layers, and wherein each of the layers has a stiffness different than that of the other layers.
Clause 70. The elongate shaft of any one of the Clauses herein, wherein the third region comprises a first layer having a first stiffness, a second layer radially peripheral to the first layer and having a second stiffness, and a third layer radially peripheral to the second layer and having a third stiffness, the second stiffness being stiffer than the first stiffness and the third stiffness being stiffer than the second stiffness.
Clause 71. The elongate shaft of any one of the Clauses herein, wherein the first region comprises at least one of a lubricious material, a polyolefin elastomer, polytetrafluoroethylene (PTFE), and/or a slip agent.
Clause 72. The elongate shaft of any one of the Clauses herein, wherein the second region comprises at least one of polymer tubing and/or braided tubing.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1A shows a partially schematic perspective view of a medical device delivery system, in accordance with embodiments of the present technology.

FIG. 1B shows a cross-sectional view of a portion of the medical device delivery system of FIG. 1B during advancement through a blood vessel, in accordance with embodiments of the present technology.

FIG. 2A shows a cross-sectional side view of an elongate shaft, in accordance with embodiments of the present technology.

FIGS. 2B and 2C show cross-sectional end views of different regions of the elongate shaft shown in FIG. 2A.

FIG. 3A shows a cross-sectional side view of an elongate shaft, in accordance with embodiments of the present technology.

FIG. 3B shows a cross-sectional end view of the elongate shaft shown in FIG. 3A.

FIG. 4 shows a flow diagram of a method for manufacturing an elongate shaft, in accordance with embodiments of the present technology.

### DETAILED DESCRIPTION

Conventional medical delivery systems often utilize a large-caliber catheter (e.g., a distal access catheter or aspiration catheter) and a small-caliber catheter (e.g., a microcatheter) disposed within the large-caliber catheter to deliver a medical device to a treatment site of a patient. The smaller inner diameter (ID) of the microcatheters, relative to the ID of the large-caliber catheter, enables delivery of certain devices (e.g., thrombectomy devices, stents, coils, etc.) to the treatment site. However, the outer diameter (OD) of the microcatheters is often much smaller than the ID of the large-caliber catheter, such that a large gap exists therebetween. As a result, “ledge effect” often occurs, which makes navigating the large-caliber catheter beyond certain branched arteries difficult. To mitigate the ledge effect associated with the use of large-caliber and small-caliber catheters in tandem, attempts have been made to instead utilize large-caliber and medium-caliber catheters. However, while such attempts can reduce the ledge effect, current medium-caliber catheters are unable to successfully deliver certain devices that require the smaller ID and necessary OD of microcatheters.

Embodiments of the present technology provide an improved catheter or elongate shaft that mitigates and/or reduces the risk of such issues. For example, as explained in additional detail elsewhere herein, embodiments of the present technology can comprise an elongate shaft comprising a lumen extending along a length thereof, and a structure surrounding the lumen that has an outermost surface tapered in the distal direction. The tapered outermost surface may constitute a longitudinal region of the elongate shaft such that, when the elongate shaft is in use, the tapered region is distal to the distal end of the large-bore catheter that the elongated shaft is partially disposed within. As such, the OD of elongate shaft at the distal end of the large-bore catheter is large enough to inhibit or reduce any ledge effect. Additionally, the elongate shaft can have an ID that is no larger than a predetermined dimension and thereby enables effective delivery of certain medical devices (previously described). In some embodiments, the structure can comprise an innermost region or layer, an outermost region or layer, and/or one or more intermediate regions or layers between the innermost and outermost regions. The structure, including its layers and/or materials, can enable the elongate shaft to have an optimal combination of flexibility, ovalization resistance, and column strength for advancing the elongate shaft through corporeal lumens of the patient and delivering devices to a treatment site.

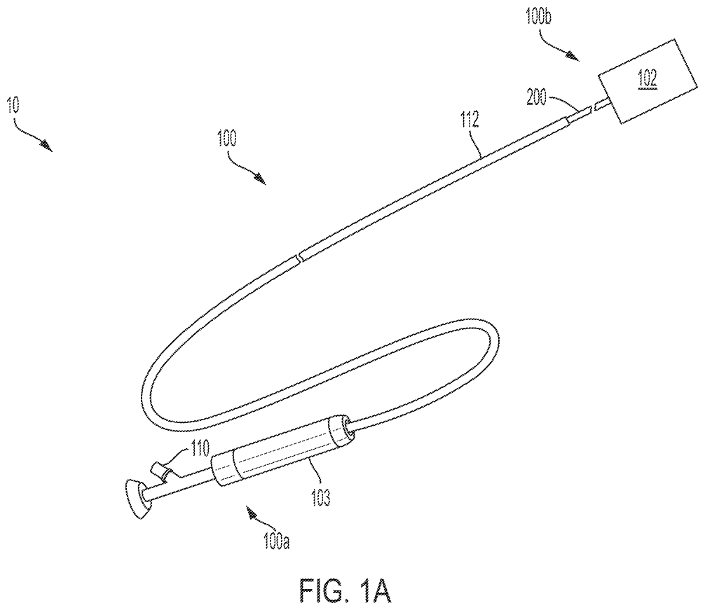

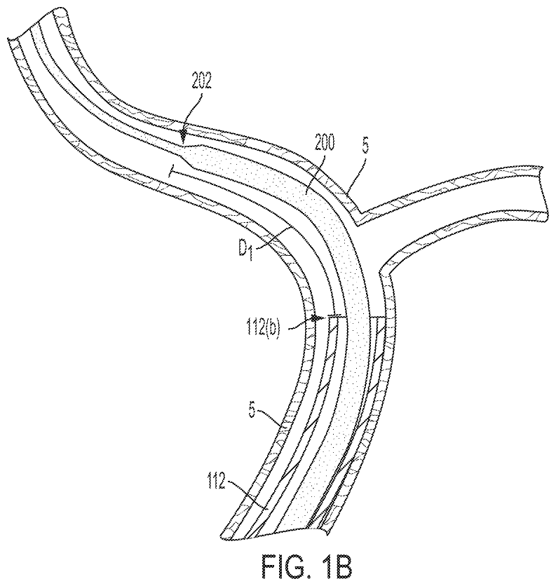

FIGS. 1A-4 depict embodiments of medical device delivery systems or portions thereof that may be used to deliver and/or deploy a medical device into a hollow anatomical structure (e.g., a blood vessel). Although many of the embodiments are described with respect to devices, systems, and methods for delivery of stents and other medical devices, other applications and other embodiments in addition to those described herein are within the scope of the present technology. Further, embodiments of the present technology can have different configurations, components, and/or procedures than those shown or described herein. Moreover, embodiments of the present technology can have configurations, components, and/or procedures in addition to those shown or described herein and these and other embodiments may not have several of the configurations, components, and/or procedures shown or described herein without deviating from the present technology.

As used herein, the terms “distal” and “proximal” define a position or direction with respect to a clinician or a clinician's control device (e.g., a handle of a delivery catheter). For example, the terms, “distal” and “distally” refer to a position distant from or in a direction away from a clinician or a clinician's control device along the length of device. In a related example, the terms “proximal” and “proximally” refer to a position near or in a direction toward a clinician or a clinician's control device along the length of device. The headings provided herein are for convenience only and should not be construed as limiting the subject matter disclosed.

### I. Overview of Systems of the Present Technology

FIG. 1A illustrates a view of a system 10 for delivery of a medical device 102, in accordance with one or more embodiments of the present technology. As shown in FIG. 1A, the system 10 comprises a delivery system 100. The delivery system 100 may comprise a medical device 102 (shown schematically) detachably coupled thereto, and may be configured to intravascularly position the device 102 at a target site within a patient. The delivery system 100 has a proximal portion 100a configured to be extracorporeally positioned during treatment, and a distal portion 100b configured to be intravascularly positioned within a blood vessel (such as an intracranial blood vessel) at or proximate a treatment site. The delivery system 100 may include a handle 103 at the proximal portion 100a, the medical device 102 at the distal portion 100b, and a plurality of elongate shafts or members extending between the proximal and distal portions 100a, 100b. In some embodiments, such as that shown in FIG. 1A, the delivery system 100 may include a first elongate shaft 112 (e.g., a guide catheter, an aspiration catheter, or a balloon guide catheter), and a second elongate shaft 200 (e.g., a delivery catheter or a microcatheter) configured to be slidably disposed within a lumen of the first elongate shaft 112.

In some embodiments, the second elongate shaft 200 is generally constructed to track over a conventional guidewire in the cervical anatomy and into the cerebral vessels associated with the brain, and may also be chosen according to several standard designs that are generally available. The second elongate shaft 200 can have a length that is at least 125 cm long, and more particularly may be between about 125 cm and about 200 cm long. In some embodiments, the second elongate shaft 200 can have any of the various lengths of the MARKSMAN™ catheter available from Medtronic Neurovascular of Irvine, Calif. USA. As described in detail elsewhere herein, the second elongate shaft 200 may have an inner diameter of about 0.015 inches (0.0381 cm), 0.017 inches (0.043 cm), about 0.021 inches (0.053 cm), or about 0.027 inches (0.069 cm). Other designs and dimensions are contemplated.

In some embodiments, the delivery system 100 may further include an elongated member (e.g., a guidewire or pushwire; not shown) configured to be slidably disposed within a lumen of the second elongate shaft 200. The elongated member can be movable within the first and/or second elongate shafts 112, 200 to position the medical device 102 at a desired location. The elongated member can be sufficiently flexible to allow manipulation, e.g., advancement and/or retraction, of the medical device 102 through tortuous passages. Tortuous passages can include, for example, catheter lumens, microcatheter lumens, blood vessels, urinary tracts, biliary tracts, and airways. The elongated member can be formed of any material and in any dimensions suitable for the task(s) for which the system is to be employed. In some embodiments, the elongated member can comprise a solid metal wire. In some embodiments, the elongated member may comprise any other suitable form of shaft such as an elongate tubular shaft.

### II. Selected Methods for Treating Aneurysms, Strokes and Other Medical Conditions

FIG. 1B shows a cross-sectional view of a portion of the delivery system 100 of FIG. 1A during advancement through a blood vessel 5. As shown in FIG. 1B, the second elongate shaft 200 has a portion disposed within the first elongate shaft 112 and another portion distal to the distal end 112b of the first elongate shaft 112. As explained in detail elsewhere herein, the second elongate shaft 200 may be tapered in a distal direction such that an outermost cross-sectional dimension (e.g., an outer diameter (OD)) at a first portion of the second elongate shaft 200 is less than an outermost cross-sectional dimension at a second, more proximal portion of the second elongate shaft 200. In some embodiments, the tapered portion (as indicated by area 202) of the second elongate shaft 200 is distal to the distal end 112b of the first elongate shaft 112. That is, when the delivery system 100 is in use and advanced within a blood vessel 5 toward a treatment site, the tapered portion 202 of the second elongate shaft 200 is distal to the distal end 112b of the first elongate shaft 112. As shown in FIG. 1B, the second elongate shaft 200 may be configured such that the tapered portion 202 is spaced apart from the distal end 112b of the first elongate shaft 112 by a distance (D), e.g., of at least about 2 centimeters (cm), 3 cm, 4 cm, or 5 cm. In some embodiments, the distance (D) (i.e., separation from the tapered portion 202 to the distal end 112b of the first elongate shaft 112) can allow the second elongate shaft 200 to pass through clots with minimal disruption to the clot and/or emboli formation. Designing or configuring the second elongate shaft 200 such that (i) the tapered portion 202 of the second elongate shaft 200 is distal to the distal end 112b of the first elongate shaft 112, and (ii) the outermost cross-sectional dimension of the second elongate shaft 200 disposed within the first elongate shaft 112 is larger than that of the tapered portion 202 can inhibit or decrease the likelihood of any ledge effect. As a result, navigation of the first and second elongate shafts past certain artery branches (e.g., the ophthalmic artery branch) may not experience any significant ledge effect. Additionally, the tapered portion 202 can enable the delivery system 100 and/or second elongate shaft 200 to delivery certain medical devices (e.g., coils, stents, etc.) that often require a small inner diameter (ID) and thus have a significantly smaller OD relative to large-bore catheters (such as the first elongate shaft 112). In some embodiments, the increased outermost cross-sectional dimension of the second elongate shaft 200 disposed within the first elongate shaft 112 can provide an optimal combination of flexibility, ovalization resistance (e.g., “kinking”), and column strength (e.g., “pushability”) for delivering medical devices to a target site.

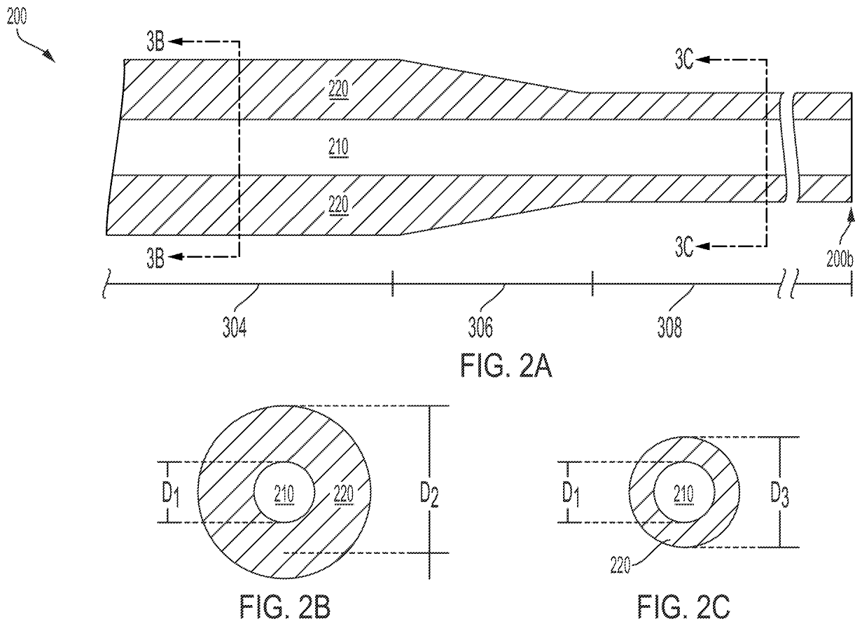

FIG. 2A shows a cross-sectional side view of an elongate shaft 200 (e.g., the second elongate shaft 200), in accordance with embodiments of the present technology. The elongate shaft 200 can include a lumen 210 extending along an entire longitudinal length of the elongate shaft 200, and a structure 220 surrounding the lumen 210 and extending along all or a portion of the length of the elongate shaft 200. In some embodiments, the lumen 210 is defined by the structure 220 in that the structure is directly proximate the lumen 210. The ID of the elongate shaft 200, e.g., a first outermost cross-sectional dimension (D₁) of the lumen 210, can be (i) at least about 0.017″, 0.021″, 0.027″, or 0.033″, (ii) no more than about 0.017″, 0.021″, 0.027″, or 0.033″, or (iii) between about 0.017″ and 0.033″.

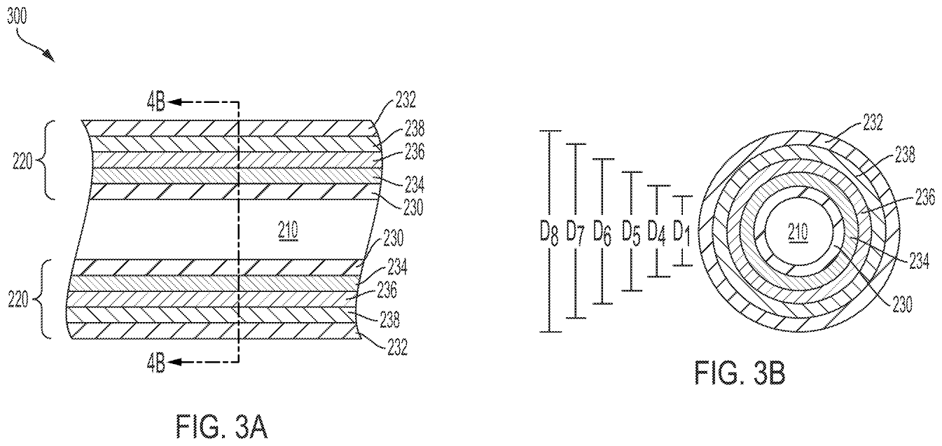

The structure 220 can comprise a single material or layer and/or multiple materials or layers, as explained in additional detail elsewhere herein (e.g., with reference to FIGS. 3A and 3B). The structure 220 can comprise Nitinol, titanium (e.g., titanium beta III), 35N LT (e.g., the 35N LT high performance alloy marketed by Fort Wayne Metals Research Products of Fort Wayne, Ind. USA), stainless steel, cobalt, chromium, drawn filled tubing (DFT), alloys thereof (e.g., cobalt-chromium), and/or combinations thereof. Additionally or alternatively, the structure 220 can comprise one or more of a lubricious material, an elastomer (e.g., polyolefin elastomer), a synthetic fluoropolymer (e.g., polytetrafluoroethylene (PTFE)), polymeric tubing, braided tubing, and/or a slip agent (e.g., a migratory slip agent, a permanent slip agent, etc.). In some embodiments where a slip agent is used, the slip agent is used in combination with a low durometer polymer, e.g., to improve lubricity. In some embodiments, the structure 220 can have a stiffness that varies in a radially outward direction (i.e., away from the lumen 210) and/or along the longitudinal length of the structure 220. For example, the stiffness profile of the structure 220 may increase or decrease in a distal direction, and/or increase or decrease in a radially outward direction.

As shown in FIG. 2A, the elongate shaft 200 can include a first portion 304 extending from a proximal end of the elongate shaft 200, a second portion 306 (e.g., a tapering portion) extending distally from the first portion 304, and a third portion 308 extending distally from the second portion 306. The elongate shaft 200 or structure 220 can be tapered such that an outermost cross-sectional dimension of the first portion 304 is greater than an outer cross-sectional dimension of the third portion 308. The second portion 306 can have an outer cross-sectional dimension that tapers in a distal direction, e.g., from the outermost cross-sectional dimension of the first portion 304 to the outer cross-sectional dimension of the third portion 308. In some embodiments, the second portion 306 is the only portion that has a tapered profile. In such embodiments, the outermost cross-sectional dimension of the first portion 304 and the second portion 306 may be substantially generally constant along a longitudinal axis of the elongate shaft 200.

The elongate shaft 200 can have a length along the longitudinal axis of at least about 100 cm or 125 cm. In some embodiments, the second portion 306 can have a length of no more than about 20 cm, 15 cm, 10 cm, 5 cm, 4 cm, 3 cm, 2 cm, 1 cm, 0.5 cm, or 0.25 cm. In some embodiments, the second portion is spaced apart from a distal end or terminus of the elongate shaft 200 or structure 220 by no more than 30 cm, 25 cm, 20 cm, 15 cm, 10 cm, 5 cm, 4 cm, 3 cm, 2 cm, or 1 cm. Additionally or alternatively, the first portion 304 can have a length generally longer than that of the catheter (e.g., first elongate shaft 112) the elongate shaft 200 is configured to be disposed within, such that the second portion 306 or tapered portion of the elongate shaft 200 can be distal to the distal end (e.g., distal end 112b) of the catheter when the catheter and elongate shaft 200 are disposed within neurovasculature of a patient. The third portion 308 may have a length generally less than that of the first portion 304.

FIGS. 2B and 2C show cross-sectional views of the respective first portion 304 and the third portion 308 of the elongate shaft 200. As shown in FIG. 2B, the first portion 304 of the elongate shaft 200 includes a second outermost cross-sectional dimension (D₂) and, as shown in FIG. 2C, the third portion 308 of the elongate shaft 200 includes a third outermost cross-sectional dimension (D₃) less than the second outermost cross-sectional dimension (D₂). The second outermost cross-sectional dimension (D₂) can be (i) at least about 1.6 French (Fr), 1.8 Fr, 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr, (ii) no more than about 1.6 Fr, 1.8 Fr, 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr, or (iii) between 1.6 Fr and 4.0 Fr. The third outermost cross-sectional dimension (D₃) can be (i) at least about 1.8 Fr, 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr, (ii) no more than about 1.8 Fr, 2.0 Fr, 2.2 Fr, 2.4 Fr, 2.6 Fr, 2.8 Fr, 3.0 Fr, 3.2 Fr, 3.4 Fr, 3.6 Fr, 3.8 Fr, or 4.0 Fr, or (iii) between 1.8 Fr and 4.0 Fr.

FIG. 3A shows a cross-sectional side view of a region of an elongate shaft 300, in accordance with embodiments of the present technology, and FIG. 3B shows a cross-sectional view of the elongate shaft 300 shown in FIG. 3A. The elongate shaft 300 can be identical to or include any of the features of the elongate structure 220 described elsewhere herein. Additionally, the region of the elongate shaft 300 shown in FIGS. 3A and 3B can correspond to any one of the first, second, or third portions 304, 306, 308 (FIG. 2A) previously described.

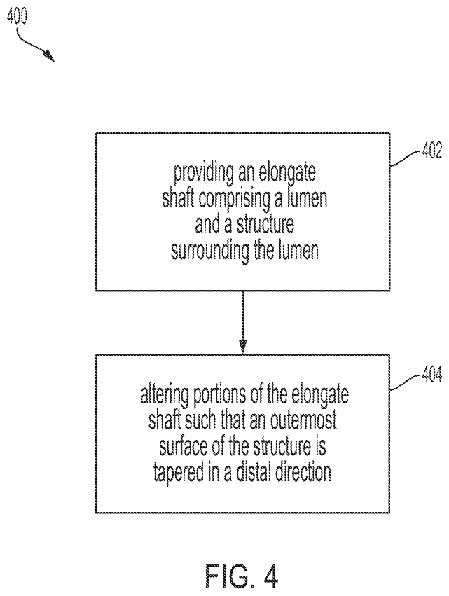

Referring to FIGS. 3A and 3B together, the elongate shaft includes the lumen 210 and the structure 220, as previously described. The structure 220 can include multiple regions (e.g., two regions, three regions, four regions, five regions, six regions, or more), which may be arranged adjacent or stacked over one another such that each region is peripheral to and/or radially outward of its adjacent region. As shown in FIG. 4A, the structure 220 can include a first region 230 (e.g., a first layer) or innermost region (e.g., an innermost layer) proximate the lumen 210, and a second region 232 (e.g., a second layer) or outermost region (e.g., an outermost layer) peripheral to and/or radially outward of the first region 230. In some embodiments, the structure 220 may include only the first and second regions 230, 232. However, in other embodiments the structure 220 may include additional intermediate regions between the first and second regions 230, 232. As shown in FIGS. 4A and 4B, for example, these additional regions can include a third region 234 (e.g., a third layer) peripheral to and/or radially outward of the first region 230, a fourth region 236 (e.g., a fourth layer) peripheral to and/or radially outward of the third region 234, and a fifth region 238 (e.g., a fifth layer) peripheral to and/or radially outward of the fourth region 236.

Each of the first, second, third, fourth, and fifth regions 230, 232, 234, 236, 238 at least partially surrounds the lumen 210 along all or a portion of the length of the elongate shaft 300. As such, each of the first, second, third, fourth, and fifth regions 230, 232, 234, 236, 238 can correspond to any one of the first, second, or third portions 304, 306, 308 (FIG. 2A) previously described. Additionally or alternatively, each of the first, second, and third portions 304, 306, 308 can include one, some, or all of the first, second, third, fourth, and fifth regions 230, 232, 234, 236, 238. For example, in some embodiments, the first portion 304 can include all of the first, second, third, fourth, and fifth regions 230, 232, 234, 236, 238, the second portion 306 can include the first, second, and third regions 230, 232, 234, and the third portion 308 can include the first and second regions 230, 232. As another example, each of the first, second, and third portions 304, 306, 308 can include each of the first, second, third, fourth, and fifth regions 230, 232, 234, 236, 238.

Each of the first, second, third, fourth, and fifth regions 230, 232, 234, 236, 238 can comprise one of more of Nitinol, titanium (e.g., titanium beta III), 35N LT, stainless steel, cobalt, chromium, alloys thereof, and/or combinations thereof. Additionally or alternatively, each of the first, second, third, fourth, and fifth regions 230, 232, 234, 236, 238 can comprise one of more of a lubricious material, elastomer (e.g., polyolefin elastomer), synthetic fluoropolymer (e.g., PTFE), drawn filled tubing (DFT), polymeric tubing, braided tubing, fiber, and/or slip agent. In some embodiments where a slip agent is used, the slip agent is used in combination with a low durometer polymer, e.g., to improve lubricity. In some embodiments, the structure 220 can have a stiffness that varies in a radially outward direction (i.e., away from the lumen 210) and/or along a longitudinal length of the structure 220. For example, the stiffness profile of the structure 220 may increase or decrease in a distal direction, and/or may increase or decrease in a radially outward direction. In some embodiments, the first or innermost region 230 can comprise a lubricious and/or hydrophilic polymeric material or coating, a low durometer polyolefin elastomer, PTFE, and/or other materials known to have lower frictional forces. The second or outermost region 232 can comprise a polymeric tubing material and/or braided tubing, which may act as a jacket along a longitudinal length of the elongate shaft 300. In some embodiments, the second region 232 can have a stiffness profile that varies (e.g., increases or decreases) along the longitudinal length of the elongate shaft 300.

The intermediate regions, e.g., the third, fourth, and fifth regions 234, 236, 238, of the structure 220 can each comprise the same of different materials as one another. Each of the intermediate regions can comprise Nitinol, titanium (e.g., titanium beta III), 35N LT, stainless steel, cobalt, chromium, cobalt-chromium, fiber, alloys thereof, and/or combinations thereof. Additionally or alternatively, each of the intermediate regions can comprise a coil (e.g., a partially coiled structure) or a braid (e.g., a partially braided structure). In those embodiments having a coil, the pitch of the loops of the coil may vary over the longitudinal length of the elongate shaft 300 or structure 220. For example, in some embodiments the pitch of the loops may decrease in a distal direction such that there is a denser loop arrangement in the proximal area of the elongate shaft 300 or structure 220 than in the distal area of the elongate shaft 300 or structure 220. Stated differently, the pitch of the loops may result in the formation of a gap between adjacent loops and the width of these gaps may increase in a distal direction such that the gap of adjacent loops at the proximal area of the elongate shaft 300 or structure 220 is smaller than the gap of adjacent loops at the distal area of the elongate shaft 300 or structure 220. In those embodiments utilizing a coiled structure, the particular pitch for each area of the structure 220 may be based on the application of the elongate shaft 300, e.g., to achieve a particular flexibility, ovalization resistance, and/or column strength of the elongate shaft 300.

The fourth region 236, or more particularly the outermost region of the intermediate regions, can include one or more materials or layers of polymeric tubing. In embodiments having multiple materials or layers, each of the materials or layers may have a stiffness that increases in a radially outward direction. That is, for an embodiment in which the fourth region 236 comprises three layers, e.g., of polymeric tubing, the first layer can have a first stiffness, the second layer can have a second stiffness stiffer than the first stiffness, and the third layer can have a third stiffness stiffer than the second stiffness. In other embodiments, the fourth region 236 may have more (e.g., four or five) or less (e.g., one or two) layers.

Each of the first, second, third, fourth, and fifth regions 230, 232, 234, 236, 238 can have an outermost cross-sectional dimension different (e.g., larger) than one or more of the other regions. For example, the first region 230 can have a fourth outermost cross-sectional dimension (D₄) greater than the first outermost cross-sectional dimension (D₁) of the lumen 210, the third region 234 can have a fifth outermost cross-sectional dimension (D₅) greater than the fourth outermost cross-sectional dimension (D₄), the fourth region 236 can have a sixth outermost cross-sectional dimension (D₆) greater than the fifth outermost cross-sectional dimension (D₅), the fifth region 238 can have a seventh outermost cross-sectional dimension (D₇) greater than the sixth outermost cross-sectional dimension (D₆), and the second region 232 can have an eighth outermost cross-sectional dimension (D₈) greater than the seventh outermost cross-sectional dimension (D₇). Each of the fourth, fifth, sixth, seventh, and eighth outermost cross-sectional dimensions (D4, D5, D6, D7, D8) can be based on the application of the elongate shaft 300, e.g., to achieve a particular flexibility, ovalization resistance, and/or column strength of the elongate shaft 300. The thickness of each of the fourth, fifth, sixth, seventh, and eighth outermost cross-sectional dimensions (D4, D5, D6, D7, D8) can be the same or different from one another.

FIG. 4 shows a flow diagram of a method 400 for manufacturing an elongate shaft, in accordance with embodiments of the present technology. The method 400 can include providing an elongate shaft (e.g., the elongate shaft 200 or 300) comprising a lumen (e.g., the lumen 210) extending along a length of the elongate shaft, and a structure (e.g., the structure 220) surrounding the lumen (process portion 402). The elongate shaft can comprise any of the features previously described herein.

The method 400 can further comprise altering portions of the elongate shaft such that an outermost surface of the structure is tapered in a distal direction (process portion 404). In some embodiments, altering portions of the elongate shaft can comprise removing the portions of the elongate shaft via chemically etching, grinding, laser ablation, skiving, or microblasting. In some embodiments, altering portions of the elongate shaft can comprise joining two or more materials together via laser welding, soldering, resistance welding, or thermal gluing.

### III. CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Although many of the embodiments are described above with respect to systems, devices, and methods for manufacturing core members for use with medical devices, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-4.

1. An elongate shaft for delivering a medical device, comprising:
a lumen extending along a length of the elongate shaft; and
a structure surrounding the lumen along the length of the elongate shaft, wherein an outermost surface of the structure is tapered in a distal direction such that an outermost cross-sectional dimension at a proximal portion of the structure is greater than an outermost cross-sectional dimension at a distal portion of the structure, and wherein the structure comprises an innermost cross-sectional dimension extending substantially along the length of the elongate shaft.

2. The elongate shaft of claim 1, wherein the structure comprises:
a first innermost region proximate the lumen; and
a second outermost region radially outward of the first region, the second region comprising the outermost surface of the structure.

3. The elongate shaft of claim 2, wherein the first region comprises at least one of a lubricious material, an elastomer, a polyolefin elastomer, a low durometer polyolefin elastomer, a synthetic fluoropolymer, polytetrafluoroethylene (PTFE), a migratory slip agent, and/or a permanent slip agent.

4. The elongate shaft of claim 2, wherein the second region comprises at least one of polymer tubing and/or braided tubing.

5. The elongate shaft of claim 2, wherein the second region comprises a stiffness that varies along at least a portion of the length of the elongate shaft.

6. The elongate shaft of claim 1, wherein the structure comprises:
a first region proximate the lumen;
a second region radially outward of the first region; and
a third region radially between the first and second regions.

7. The elongate shaft of any claim 6, wherein the third region comprises a coiled or helical structure.

8. The elongate shaft of claim 6, wherein the third region comprises a coiled structure having a coil pitch that varies along the length of the elongate shaft.

9. The elongate shaft of claim 1, wherein an innermost cross-sectional dimension of the elongate shaft is substantially constant along a distal portion of the elongate shaft.

10. The elongate shaft of claim 1, wherein the innermost cross-sectional dimension of the elongate shaft is substantially constant along the entire length of elongate shaft.

11. An elongate shaft for delivering a medical device, comprising:
a lumen extending along a length of the elongate shaft; and
a structure surrounding the lumen along the length of the elongate shaft, the structure comprising a first portion extending longitudinally from a proximal portion of the structure, a second portion extending longitudinally from the first portion, and a third portion extending longitudinally from the second portion to a distal portion of the structure, wherein an outermost surface of the structure is tapered in a distal direction such that an outermost cross-sectional dimension at the proximal portion of the structure is greater than an outermost cross-sectional dimension at the distal portion of the structure, and wherein the outermost cross-sectional dimension at the distal portion of the structure is the minimum outermost cross-sectional dimension of the structure.

12. The elongate shaft of claim 11, wherein the structure comprises:
a first region proximate the lumen and extending longitudinally along the length of the elongate shaft;
a second region radially outward of the first region and extending longitudinally along the length of the elongate shaft; and
a third region radially between the first and second regions and extending longitudinally along the length of the elongate shaft.

13. The elongate shaft of claim 12, wherein the first region, second region, and third region each comprise a layer, and wherein each of the first region, second region, and third region have a tapered portion.

14. The elongate shaft of claim 11, wherein the structure comprises:
a first region proximate the lumen;
a second region radially outward of the first region;
a third region radially between the first and second regions; and
a fourth region radially between the second and third regions.

15. The elongate shaft of claim 14, wherein the fourth region comprises a stiffness that varies in a distal direction.

16. The elongate shaft of claim 14, wherein the fourth region comprises a first layer having a first stiffness, a second layer radially peripheral to the first layer and having a second stiffness, and a third layer radially peripheral to the second layer and having a third stiffness, the second stiffness being stiffer than the first stiffness and the third stiffness being stiffer than the second stiffness.

17. An elongate shaft for delivering a medical device, comprising:
a lumen extending along a length of the elongate shaft and having a substantially constant cross-sectional dimension along the length; and
a structure surrounding the lumen along the length of the elongate shaft, the structure comprising a first region extending longitudinally along the length of the elongated shaft, a second region radially outward of the first region, and a third region radially outward of the second region,
wherein an outermost surface of the structure is tapered in a distal direction such that an outermost cross-sectional dimension at a proximal portion of the structure is greater than an outermost cross-sectional dimension at a distal portion of the structure, and
wherein the outermost cross-sectional dimension at the distal portion of the structure is the minimum outermost cross-sectional dimension of the structure.

18. The elongate shaft of claim 17, wherein the third region comprises a stiffness that varies in a distal direction.

19. The elongate shaft of claim 17, wherein the third region comprises a plurality of layers, and wherein each of the layers has a stiffness different than that of the other layers.

20. The elongate shaft of claim 17, wherein the third region comprises a first layer having a first stiffness, a second layer radially peripheral to the first layer and having a second stiffness, and a third layer radially peripheral to the second layer and having a third stiffness, the second stiffness being stiffer than the first stiffness and the third stiffness being stiffer than the second stiffness.
